(19) United States
(12) Patent Application Publication
(10) Pub. No.: US 2007/0292882 A1
(43) Pub. Date: Dec. 20, 2007

(54) Method and apparatus for detecting cancer cell

(75) Inventors: Hideki Takata, Kobe (JP); Kayo Hiyama, Kobe (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 11/808,488

(22) Filed: Jun. 11, 2007

(30) Foreign Application Priority Data
Jun. 16, 2006 (JP) 2006-168131

(51) Int. Cl. C12Q 1/68; C12M 3/00

(52) U.S. Cl. 435/6; 435/287.2

(57) ABSTRACT

A method for determining a cancer cell which has good precision and high reliability, including a step of quantitating an mRNA transcribed from a tumor marker gene contained in a biological sample, a step of quantitating a polypeptide translated from the tumor marker gene in the biological sample, and a step of detecting a cancer cell in the biological sample based on the quantitation result of the mRNA and the quantitation result of the polypeptide.

### FIELD OF THE INVENTION

The present invention relates to a method and an apparatus for detecting a cancer cell contained in a biological sample.

### BACKGROUND

Conventionally, in order to detect a cancer cell in a sample collected from a living body (biological sample), an expression amount of a tumor marker gene in a biological sample has been measured. When an expression amount of a tumor marker gene is greater (or smaller) than an expression amount of a normal cell, it is suspected that a cancer cell is contained in the biological sample.

By detecting a cancer cell in a biological sample as described above, finding of a cancer in the biological sample, and determination of the presence or absence of metastasis of a cancer to the biological sample can be performed. For example, U.S. Pat. No. 5,766,888 discloses a method for determining metastasis of a cancer by extracting and purifying an RNA sample from a biological sample such as a body tissue and a body fluid, and determining whether a cancer-associated sequence (mRNA of tumor marker gene) is contained in this RNA sample or not.

### SUMMARY

The scope of the present invention is defined solely by the appended claims, and is not affected to any degree by the statements within this summary.

An object of the present invention is to provide a method for detecting a cancer cell, which has better precision and higher reliability than those of the aforementioned conventional method for detecting a cancer cell based on an expression amount of an mRNA.

The present invention provides a method for detecting a cancer cell, comprising a step of quantitating an mRNA transcribed from a tumor marker gene contained in a biological sample, a step of quantitating a polypeptide translated from the tumor marker gene in the biological sample, and a step of detecting a cancer cell in the biological sample based on the result of quantitation of the mRNA and the result of quantitation of the polypeptide.

Thereby, a method for detecting a cancer cell at better precision and higher reliability can be provided.

In the detection method, it is preferable that the biological sample is a lymph node tissue.

Thereby, whether a cancer cell has been metastasized to a lymph node or not can be determined.

In the detection method, it is preferable that quantitation of the mRNA and quantitation of the polypeptide are performed using the same sample for detection.

Thereby, it is not required that a sample for quantitating an mRNA and a sample for quantitating a polypeptide are separately prepared. Therefore, a cancer cell can be detected easily.

Also, the present invention provides a method for detecting a cancer cell, comprising a step of detecting an mRNA transcribed from a tumor marker gene, contained in a sample for detection prepared by mixing a biological sample and a buffer, a step of detecting a polypeptide translated from the tumor marker gene, contained in the sample for detection, and a step of detecting a cancer cell in the biological sample based on the result of quantitation of the mRNA and the result of quantitation of the polypeptide.

As used herein, detection includes quantitative determination of whether a molecule (mRNA and polypeptide) to be measured is contained in a sample for detection or not, and quantitation of this molecule.

Thereby, it is not required that a sample for measuring an mRNA and a sample for quantitating a polypeptide are separately prepared. Therefore, a cancer cell can be detected easily.

Also, the present invention provides a method for preparing a sample for detection for detecting a nucleic acid and a polypeptide, comprising treating a biological sample using a buffer of a pH 2.5 to 5.0 containing dimethyl sulfoxide and a surfactant, thereby, transferring a nucleic acid and a polypeptide contained in the biological sample to a solution to prepare a sample for detection.

By using this method, a sample for detection containing a nucleic acid and a polypeptide can be prepared. For this reason, measurement of a nucleic acid and measurement of a polypeptide can be performed from the same sample for detection.

Therefore, it is not required that a sample for measuring a nucleic acid and a sample for measuring a polypeptide are separately prepared. Accordingly, a nucleic acid and a polypeptide can be measured easily.

Also, the present invention provides an apparatus for detecting a cancer cell, comprising a first quantitation part for quantitating an mRNA transcribed from a tumor marker gene contained in a biological sample, a second quantitation part for quantitating a polypeptide translated from the tumor marker gene contained in the biological sample, and a determination part for detecting a cancer cell in the biological sample based on the result of quantitation of the mRNA and the result of quantitation of the polypeptide.

Thereby, an apparatus which can detect a cancer cell at high reliability and better precision can be provided.

### BRIEF DESCRIPTION OF THE DRAWINGS

FIG. 1 is a perspective view showing an entire construction of a cancer cell detection apparatus 1 in accordance with one embodiment of the present invention;

FIG. 2 is a perspective view showing an entire construction of a nucleic acid quantitation part 100;

FIG. 3 is a schematic plane view of the nucleic acid quantitation part of FIG. 2;

FIG. 4 is a perspective view showing an entire construction of a protein quantitation part 101;

FIG. 5 is a view for explaining a principle of protein quantitation with the protein quantitation part 101;

FIG. 6 is a view for explaining a principle of protein quantitation with the protein quantitation part 101;

FIG. 7 is a treatment flow with CPU102d; and

FIG. 8 is a graph showing the result of Example 1.

FIG. 9 is a graph showing the result of Example 2.

FIG. 10 is a graph showing the result of Example 3.

### DETAILED DESCRIPTION OF THE EMBODIMENT

A method for detecting a cancer cell which is one embodiment of the present invention is a method for detecting a cancer cell in a biological sample containing a plurality of cells. This method comprises a first quantitation step of quantitating an mRNA transcribed from a predetermined tumor marker gene, a second quantitation step of quantitating a polypeptide translated from this gene, and a detection step of detecting a cancer cell in the biological sample based on the result of quantitation of the mRNA and the result of quantitation of the polypeptide.

As used herein, a cancer is a tumor which has became malignant, and has the same meaning as a malignant tumor. The cancer includes carcinoma, sarcoma, and a cancer derived from a hematopoietic organ. As the carcinoma, an epithelial cell-derived cancer such as a breast cancer, a stomach cancer, a colon cancer, a prostate cancer, a cervical cancer, and a cancer of uterine body is exemplified. As the sarcoma, osteosarcoma and soft-tissue sarcoma are exemplified. As the cancer derived from a hematopoietic organ, leukemia and malignant lymphoma are exemplified.

The biological sample is not particularly limited, as far as it is a sample containing a plurality of cells collected from an animal such as human. Examples include excretion such as urine and feces, blood, and a tissue collected by a surgical procedure such as biopsy and isolation operation. Particularly, a tissue collected by biopsy is preferable.

For example, when the method is practiced using a tissue, whether this tissue has been transformed into a cancer or not can be determined. In addition, when primary focus is recognized in another tissue, whether a cancer cell has been metastasized from the primary focus to this tissue or not can be determined.

This determination result can be used as one of indices upon determination of therapeutic policy and a dissection region of a tissue. For example, collection of a lymph node tissue in the vicinity of a tumor from a breast cancer patient by biopsy, and determination of the presence or absence of metastasis of a cancer to a lymph node by the above method assist determination of whether a lymph node should be isolated, or to what extent a lymph node should be dissected.

In the first quantitation step, an mRNA transcribed from a tumor marker gene contained in the biological sample is quantitated. The tumor marker gene is a gene such that an expression amount in a cancer cell, and an expression amount in a normal cell are significantly different, as described above. The tumor marker gene is different depending on a biological sample used, and a kind of a cancer. Examples of the tumor marker gene include genes encoding CK (cytokeratin) such as CK18, CK19 and CK20, CEA (carcinoembryonic antigen), MUC1, MMG (mammaglobin), PSA (prostate specific antigen), CA15-3, EpCAM (epithelial cellular adhesion molecule), and the like.

Upon quantitation of an mRNA, it is preferable to prepare a sample for detection from the biological sample, and quantitate an mRNA contained in this sample for detection. For example, a biological sample and a buffer are mixed, cells in the buffer are chemically and/or physically treated to transfer an RNA in cells to a solution, and the solution containing the RNA can be used as a sample for detection.

It is preferable that a buffer is strongly acidic in order to suppress degradation of an RNA. A range of a pH is preferably 2.5 to 5.0, more preferably 3.0 to 4.0. In order to keep a pH in this range, a known buffer can be used.

In addition, it is preferable that the buffer contains a surfactant. A cell membrane and a nuclear membrane are damaged with the surfactant. Therefore, it becomes easy for a nucleic acid in cells to transfer into a solution through this damage. The kind of the surfactant is not particularly limited as far as it has such action. A preferable surfactant is a nonionic surfactant. A polyoxyethylene nonionic surfactant is more preferable. Particularly, a polyoxyethylene nonionic surfactant represented by the general formula:

R₁-R₂—(CH₂CH₂O)ₙ—H

(wherein R₁ is an alkyl group, an alkenyl group, an alkynyl group, or an isooctyl group having 10 to 22 carbon atoms; R₂ is —O— or —(C₆H₆)—O—; n is an integer of 8 to 120) is suitable.

Specific examples of the polyoxyethylene nonionic surfactant include polyoxyethylene lauryl ether, polyoxyethylene cetyl ether, polyoxyethylene oleyl ether, polyoxyethylene myristyl ether, polyoxyethylene stearyl ether, polyoxyethylene nonyl phenyl ether, and polyoxyethylene isooctyl phenyl ether.

More specifically, Brij35(polyoxyethylene(35)lauryl ether) and the like are suitable. A concentration of the surfactant in the buffer is preferably 0.1 to 6% (v/v), more preferably 1 to 5% (v/v).

In addition, when quantitation of an mRNA is performed by nucleic acid amplification described later, it is preferable that the buffer further contains dimethyl sulfoxide (DMSO). Although a substance inhibiting an enzymatic reaction in nucleic acid amplification (inhibitory substance) is contained in the biological sample in some cases, influence of this inhibitory substance can be effectively reduced by the action of DMSO. In addition, DMSO also has the effect of suppressing reduction in the activity of a nucleic acid amplification enzyme. A concentration of DMSO in the buffer is preferably 1 to 50% (v/v), more preferably 5 to 30% (v/v), most preferably 1 to 25% (v/v).

Upon preparation of the sample for detection, it is preferable that the biological sample is subjected to physical treatment such as homogenization. Thereby, a cell membrane and a nuclear membrane of cells in the biological sample are physically ground, and it becomes easy for a nucleic acid in cells to transfer into a solution. Homogenization may be performed manually with a pestle, or may be performed using a commercially available electric-powered homogenizer. A cell piece floating in a homogenate can be precipitated by centrifuging the resulting homogenate for a few seconds to a few minutes. Thereby, a supernatant containing an RNA and the like can be used as the sample for detection.

Quantitation of an mRNA can be performed by a known method using nucleic acid amplification, a DNA chip or the like. When a nucleic acid amplification method is used, an RT-PCR (Reverse Transcription PCR) method and an RT-LAMP (Reverse Transcription LAMP: for LAMP method, see U.S. Pat. No. 6,410,278) method comprising a reverse transcription reaction are suitably used before the nucleic acid amplification method. Particularly, as a quantitative nucleic acid amplification method, known methods such as an SYBR Green method and a TaqMan (registered trademark of Roche Diagnostics) method (see Linda G. Lee, 1993, Nucleic Acids Research, vol. 21, p3761-3766 etc.) can be used.

As the DNA chip which can be used in quantitating an mRNA, a substrate on which a polynucleotide of a DNA capable of hybridizing with a cDNA of the tumor marker gene and/or a fragment thereof is immobilized, can be used. Detection of an RNA using the DNA chip can be performed by a known method which is generally used.

For example, detection of an RNA using the DNA chip can be performed as follows. First, a reverse transcription reaction is performed utilizing a polyA sequence present on a 3′ end of an mRNA in a sample for detection. Upon the reverse transcription reaction, for example, by using a nucleotide labeled with a fluorescent substance such as Cy3 and Cy5, a fluorescently labeled cDNA is synthesized. This is contacted with the substrate on which a polynucleotide is immobilized. Thereby, this polynucleotide and the labeled cDNA form a double strand. After formation of the double strand, an mRNA can be quantitated by measuring fluorescence of the cDNA.

A quantitated value of an mRNA, measured by the above method, may be a substance amount of an mRNA, an mRNA mass, or a copy number per unit volume. Alternatively, it may be a time or a cycle number (when PCR is used) until a fluorescent intensity or a turbidity of a reaction solution reaches a predetermined value.

In the second quantitation step, a polypeptide translated from the tumor marker gene is quantitated. The polypeptide may be a protein translated from the tumor marker gene, or a fragment thereof.

Upon quantitation of the polypeptide, it is preferable that a sample for detection is prepared from the biological sample, and a polypeptide contained in this sample for detection is quantitated. This sample for detection may be a sample for detecting a polypeptide different from the sample for detection used upon quantitation of an mRNA. Preferably, the same sample as the sample for detection, prepared upon quantitation of an mRNA, is used. Thereby, labor for preparing two kinds of samples, a sample for quantitating an mRNA and a sample for quantitating a polypeptide, can be omitted. Therefore, the sample can be prepared more easily and in a short time.

Conventionally, upon quantitation of an mRNA, treatment such as extraction and purification was repeated on a biological sample using an RNA extraction kit or the like. Thereby, a sample substantially containing only an RNA was prepared, and this was used as a sample for detection. When this treatment is performed, a polypeptide in the biological sample and a piece of ground cells are discarded. Therefore, in order to quantitate a polypeptide from the same biological sample, it was necessary to prepare again a sample for detection for quantitating a polypeptide.

However, a sample for detecting an mRNA, which was prepared using a buffer having the aforementioned pH and containing the aforementioned surfactant, contains a polypeptide of a tumor marker gene. For this reason, quantitation of an mRNA and quantitation of a polypeptide can be performed using the same sample.

Quantitation of the polypeptide can be performed by a known method, being not limiting. For example, an analysis method using a protein chip or the like, an immunoblot method such as a dot blot method and a Western blot method, radioimmunoassay (RIA), enzyme immunoassay (EIA), fluorescent immunoassay (FIA), chemical emission immunoassay (CLIA), counting immunoassay (CIA: see Sysmex Journal Vol. 20 No. 1, 77-86 (1997)) can be used.

Among them, when the Western blot method is used, the kind of a polypeptide contained in a sample for detection can be separated based on a molecular weight. For this reason, an objective polypeptide can be quantitated more specifically.

It is preferable that a quantitated value of a polypeptide is calculated based on a calibration curve which was produced in advance. The calibration curve can be produced by measurement under the same condition as that of measurement of a polypeptide of a sample for detection, using a sample containing a known amount of a protein.

Based on the quantitation result of an mRNA and the quantitation result of a polypeptide measured as described above, a cancer cell in the biological sample is detected. In this step, whether a cancer cell is contained in the biological sample or not is predicted. A cancer cell may be detected based on a combination of both quantitation results. Alternatively, determination may be first performed based on the quantitation result of one of an mRNA and a polypeptide, and the result of the determination may be confirmed using the other quantitation result. The determination result obtained based on both quantitation results of an mRNA and a polypeptide has higher precision and higher reliability than those of a determination result using either one of quantitation results.

A cancer cell in a biological sample may be detected based on the comparison result obtained by comparing a quantitated value of an mRNA with a corresponding first threshold, and the comparison result obtained by comparing a quantitated value of a polypeptide with a corresponding second threshold. For example, in the case of a tumor marker gene in which an expression amount is greater in a cancer cell than in a normal cell, when a quantitated value of an mRNA is not smaller than a first threshold, and a quantitated value of a polypeptide is not smaller than a second threshold, it can be predicted that a cancer cell is contained in a biological sample. In addition, when a quantitated value of an mRNA is not smaller than a first threshold, or a quantitated value of a polypeptide is not smaller than a second threshold, it may be predicted that a cancer cell is contained in a biological sample.

The first threshold and the second threshold are values which are conveniently set depending on a kind of a cancer and that of a tumor marker. These thresholds can be set at a value being not larger than an amount of a tumor marker contained in a biological sample, for which the presence of a cancer cell has been confirmed (positive sample), and higher than an amount of a tumor marker contained in a biological sample, for which the absence of a cancer cell has been confirmed (negative sample). Particularly, it is preferable that a value obtained by measuring tumor marker amounts of a plurality of positive samples and tumor marker amounts of a plurality of negative samples in advance, by which a positive sample and a negative sample can be discriminated at a highest probability, is set as a threshold.

Alternatively, an mRNA transcribed from a tumor marker gene contained in a sample for detection prepared from the buffer is qualitatively detected, a polypeptide translated from the tumor marker gene contained in this sample for detection is qualitatively detected and, based on these detection results, a cancer cell in a biological sample may be detected. In this case, for example, when it is determined that an mRNA is present and a polypeptide is present in the sample for detection, it is determined that a cancer cell is contained in a biological sample.

A method for qualitatively detecting an mRNA is not particularly limited, but a known method can be used. For example, detection can be performed by conducting agarose gel electrophoresis or the like using a reaction solution from nucleic acid amplification by the aforementioned RT-PCR or RT-LAMP. In addition, a method for qualitatively detecting a polypeptide is also not particularly limited, but the aforementioned known immunoassay and the like can be used.

Another embodiment of the present invention is a cancer cell detection apparatus for implementing the aforementioned method. Based on the drawings, this apparatus will be explained below.

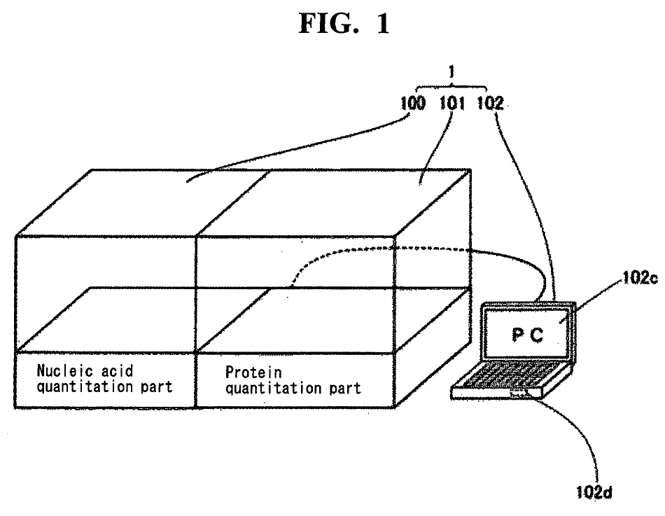

FIG. 1 is a perspective view showing an entire construction of a cancer cell detection apparatus 1 in accordance with one embodiment of the present invention. This apparatus 1 is constructed of a nucleic acid quantitation part 100, a protein quantitation part 101, and a personal computer (PC) 102 which is connected to them so that it can be communicated therewith wired or wireless. The personal computer 102, as shown in FIG. 1, comprises a display part 102c consisting of a monitor, and a CPU 102d which analyzes the result of sample measurement.

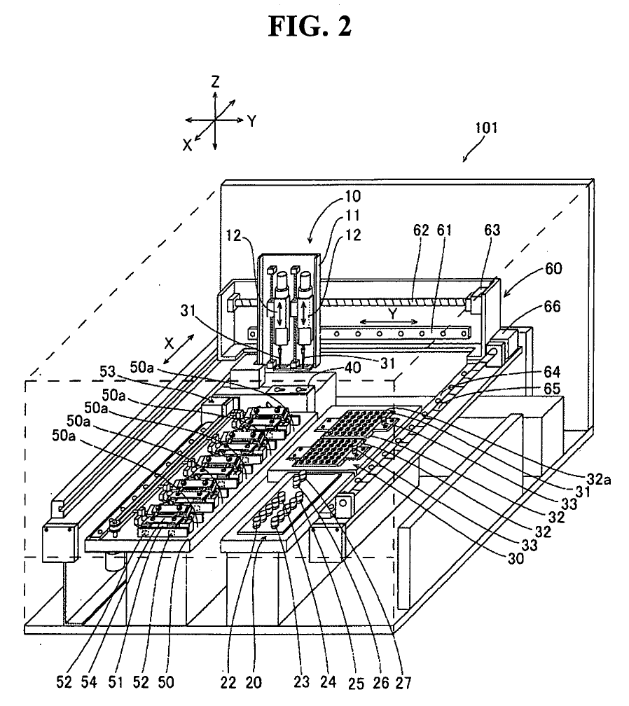

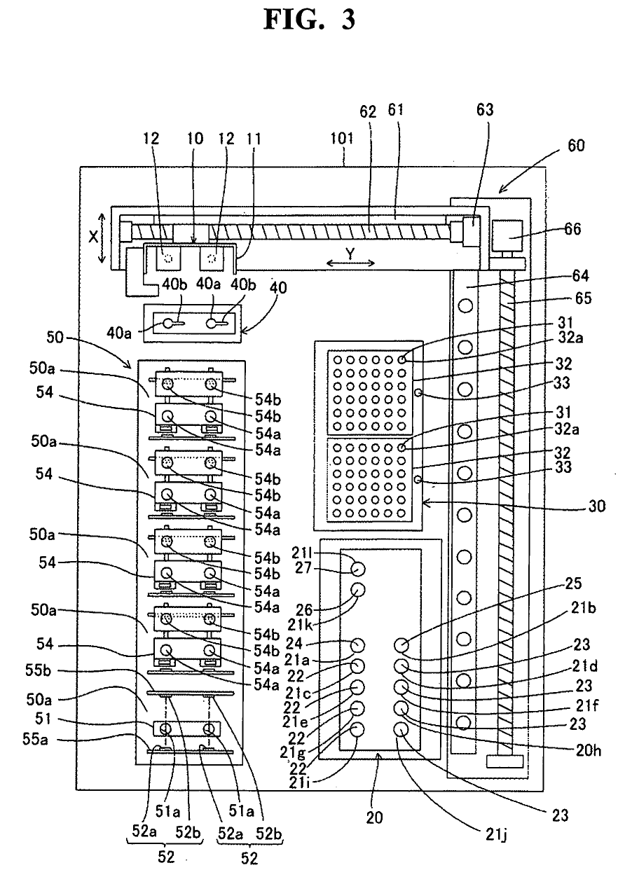

The nucleic acid quantitation part 100 of the apparatus 1 will be explained using FIG. 2 and FIG. 3. FIG. 2 is a perspective view showing an entire construction of the nucleic acid quantitation part 100. FIG. 3 is a schematic plane view of the nucleic acid quantitation part of FIG. 2.

The nucleic acid quantitation part 100, as shown in FIG. 2, comprises a dispensing mechanism part 10, a sample setting part 20, a chip setting part 30, a chip discarding part 40, a reaction detection part 50 consisting of five reaction detection blocks 50a, and a transferring part 60 for transferring the dispensing mechanism part 10 in an X axis direction and a Y axis direction.

In addition, the dispensing mechanism part 10, as shown in FIG. 2, comprises an arm part 11 which is moved by the transferring part 60 in an X axis direction and a Y axis direction (horizontal direction), and duplicate (two) syringe parts 12 which each can be independently transferred relative to the arm part 11 in a Z axis direction (vertical direction).

In addition, as shown in FIG. 2 and FIG. 3, the sample setting part 20 is provided with ten sample container setting pores 21a to 21j, one enzyme reagent container setting pore 21kand one primer reagent container setting pore 211 in an order from a front of the apparatus. In addition, the ten sample container setting pores 21a to 21j are provided so as to be arranged in five rows and two columns. In addition, the sample container setting pores 21c and 21d, the sample container setting pores 21e and 21f, the sample container setting pores 21g and 21h, and the sample container setting pores 21i and 21jare provided at a sample setting position 1, a sample setting position 2, a sample setting position 3 and a sample setting position 4, respectively, in an order from an inner side of the apparatus.

In addition, in the present embodiment, a sample container 22 accommodating an extract (sample for detection), which is prepared by solubilizing a pre-excised biological tissue with the aforementioned treatment (homogenization, filtration etc.) is set in the sample container setting pores 21c , 21e, 21g and 21i on a front left side. Furthermore, a sample container 23 accommodating a diluted sample obtained by 10-fold diluting the sample is set in the sample container setting pores 21d, 21f, 21h and 21j on a front right side.

In addition, a container 24 accommodating a positive control for confirming that a nucleic acid to be amplified is normally amplified is mounted in the sample container setting pore 21a. Furthermore, a container 25 accommodating a negative control for confirming that a nucleic acid not to be amplified is not normally amplified is set in the sample container setting pore 21b.

An enzyme reagent container 26 accommodating a nucleic acid amplification enzyme reagent for amplifying a cDNA (hereinafter, also referred to as CK19cDNA) corresponding to an mRNA of CK19 (hereinafter, also referred to as CK19 mRNA), and a primer reagent container 27 accommodating a reagent comprising a primer (hereinafter, referred to as primer reagent), which can be hybridized with CK19cDNA are set in the enzyme reagent container setting pore 21k and the primer reagent container setting pore 211, respectively.

In addition, each reaction detection block 50a of the reaction detection part 50, as shown in FIG. 2 and FIG. 3, is constructed of a reaction part 51, two turbidity detection parts 52, and a lid closing mechanism 53 (see FIG. 3). In the reaction part 51 provided in each reaction detection block 50a, as shown in FIG. 3, two detection cell setting pores 51a for setting a detection cell 54 are provided. Each reaction detection block 50a is arranged at a cell setting position 1, a cell setting position 2, a cell setting position 3, a cell setting position 4 and a cell setting position 5 in an order from an inner side of the apparatus.

In addition, the turbidity detection part 52 is constructed of an LED light source part 52a consisting of blue LED having a wavelength of 465 nm attached to a substrate 55a arranged on one side face side of the reaction part 51, and a photodiode light receiving part 52b attached to a substrate 55b arranged on the other side face side of the reaction part 51. In each reaction detection block 50a, two sets of the turbidity detection part 52 are arranged, one set consisting of one LED light source part 52a and one photodiode light receiving part 52b.

In addition, the detection cell 54 has two cell parts 54a for accommodating a sample, and a lid part 54b for covering the two cell parts 54a.

In addition, the transferring part 60, as shown in FIG. 2, comprises a direct-acting guide 61 and a ball screw 62 for transferring the dispensing mechanism part 10 in a Y axis direction, a stepping motor 63 for driving the ball screw 62, a direct-acting guide 64 and a ball screw 65 for transferring the dispensing mechanism part 10 in an X axis direction, and a stepping motor 66 for driving the ball screw 65. The dispensing mechanism part 10 is transferred in an X axis direction and a Y axis direction by rotating ball screws 62 and 65, respectively, by stepping motors 63 and 66.

Next, referring to FIG. 1 to FIG. 3, motion of the nucleic acid quantitation part 100 in accordance with the present embodiment will be explained. In this embodiment, as described above, a cDNA corresponding to CK19 mRNA (tumor marker) in a tissue excised by operation is amplified using the RT-LAMP method, and a change in a turbidity due to clouding of magnesium pyrophosphate generated accompanied with amplification is measured, thereby, an amount of CK19 mRNA (copy number/μL) is measured, and this is compared with a threshold.

First, as shown in FIG. 2 and FIG. 3, a sample container 22 accommodating a sample for detection (hereinafter, also referred to as sample) prepared by treating (homogenizing, filtering, or the like) an excised tissue in advance is set in sample container setting pores 21c to 21j. In addition, a container 24 accommodating a positive control, and a container 25 accommodating a negative control are set in sample container setting pores 21a and 21b, respectively (see FIG. 3). In addition, an enzyme reagent container 26 accommodating a nucleic acid amplification enzyme reagent for amplifying a CK19cDNA, and a primer reagent container 27 accommodating a primer reagent for amplifying a CK19cDNA are set, respectively, in an enzyme reagent container setting pore 21k (see FIG. 3) and a primer reagent container setting pore 211. In addition, two racks 32 accommodating 36 disposable pipette chips 31 are arranged in the chip setting part 30.

When motion of the nucleic acid quantitation part 100 is started, first, after the arm part 11 of the dispensing mechanism part 10 is moved from an initial position to the chip setting part 30 with the transferring part 60 shown in FIG. 2 and, at the chip setting part 30, two syringe parts 12 of the dispensing mechanism part 10 are moved downwardly. Thereby, since tips of nozzle parts of two syringe parts 12 are pressed into upper openings of two pipette chips 31, pipette chips 31 are automatically fitted into tips of nozzle parts of two syringe parts 12. Then, after two syringe parts 12 are moved upwardly, the arm part 11 of the dispensing mechanism part 10 is moved towards above the primer reagent container 27 accommodating the primer reagent in an X axis direction. Then, after one syringe part 12 situated above the primer reagent container 27 is moved in a down direction, and the primer reagent is sucked, the one syringe part 12 is moved in an upper direction. Thereafter, the arm part 11 of the dispensing mechanism part 10 is moved with the transferring part 60 in a Y axis direction, so that the other syringe part 12 is situated above the same primer reagent container 27. Then, after the other syringe part 12 is moved downwardly, and the primer reagent is sucked from the same primer reagent container 27, the other syringe part 12 is moved in an upper direction. By doing like this, the primer reagent in the primer reagent container 27 is sucked with two pipette chips 31 fitted into the syringe parts 12.

After suction of the primer reagent, after two syringe parts 12 are moved upwardly, the arm part 11 of the dispensing mechanism part 10 is moved to above the reaction detection block 50a situated at the cell setting position 1 which is an innermost side (front inner side of the apparatus), with the transferring part 60. Then, at the reaction detection block 50a on the innermost side, by moving two syringe parts 12 downwardly, two pipette chips 31 fitted into two syringe parts 12 are inserted into two cell parts 54a of the detection cell 54, respectively. Then, using the syringe parts 12, primer reagents are discharged into two cell parts 54a, respectively.

After discharge of the primer reagents, after the two syringe parts 12 are moved upwardly, the arm part 11 of the dispensing mechanism part 10 is moved towards above the chip discarding part 40 in an X axis direction with the transferring part 60. Then, at the chip discarding part 40, the pipette chip 31 is discarded. Specifically, by moving two syringe parts 12 downwardly, pipette chips 31 are inserted into two chip discarding pores 40a (see FIG. 3) of the chip discarding part 40. Under this state, by transferring the arm part 11 of the dispensing mechanism part 10 in a Y axis direction with the transferring part 60, the pipette chips 31 are moved to below a groove part 40b. Then, since collar parts on the upper sides of the pipette chips 31 are abutted against undersides on both sides of a groove part 40b, and receive a downward force from undersides thereof by moving the two syringe parts 12 upwardly, the pipette chips 31 are automatically detached from nozzle parts of two syringe parts 12. Thereby, the pipette chips 31 are discarded into the chip discarding part 40.

Then, by a similar motion, an enzyme reagent is discharged to the cell part 54a from an enzyme reagent container 26 and, further, by a similar motion, samples are discharged to the cell part 54a from a sample container 22 and a sample container 23.

Then, after the primer reagent, the enzyme reagent and the sample are discharged into the cell part 54a, motion of closing a lid of a lid part 54b of the detection cell 54 is performed. After completion of this lid closing motion, by elevating a liquid temperature in the detection cell 54 from about 20° C. to about 65° C., a cDNA corresponding to a CK19 mRNA is amplified by an RT-LAMP reaction. Then, clouding due to magnesium pyrophosphate produced accompanied with amplification is detected by nephelometry. Specifically, using an LED light source part 52a and a photodiode light receiving part 52b shown in FIG. 3, a turbidity in the detection cell 54 at an amplification reaction is detected (monitored), thereby, a turbidity is detected.

Turbidity data of the sample is sent to the personal computer 102 from the nucleic acid quantitation part 100 in real time. CPU102d of the personal computer 102 calculates an mRNA copy number per unit volume from turbidity data of the sample, and compares this with a predetermined threshold.

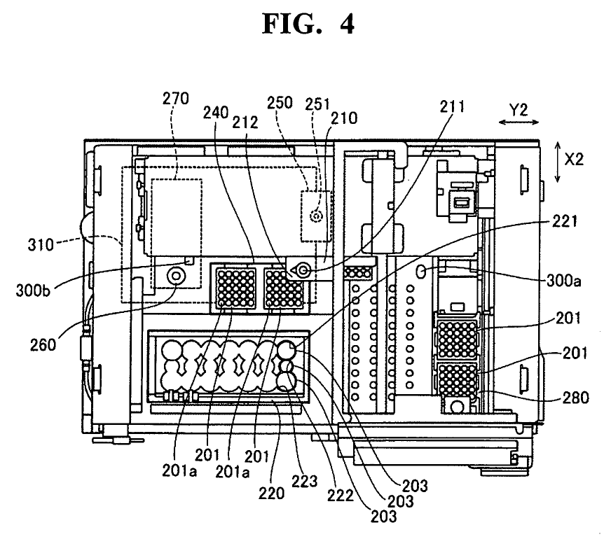

Then, based on FIGS. 4 to 6, the protein quantitation part 101 will be explained. At the protein quantitation part 101, an amount of a CK19 protein contained in the sample which has been subjected to measurement at the nucleic acid quantitation part 100 can be measured.

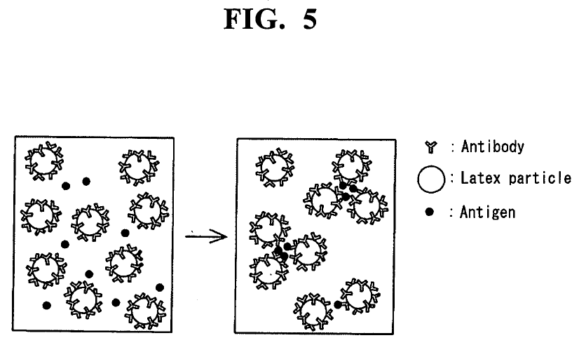

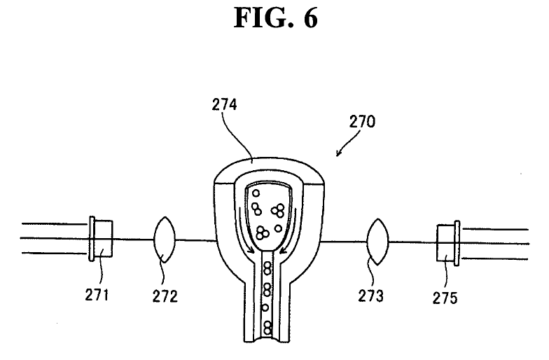

FIG. 4 is a perspective view showing an entire construction of the protein quantitation part 101, and FIGS. 5 and 6 are views for explaining principle of protein quantitation at the protein quantitation part 101.

The protein quantitation part 101, as shown in FIG. 4, comprises a dispensing part 210, a reagent setting part 220, a reaction part 240, a measurement dilution dispensing part 250, a sample receiving part 260, an optical detection part 270, a reaction plate tray 280 for accommodating an unused reaction plate 201, washing parts 300a and 300b, and a control part 310.

The dispensing part 210, as shown in FIG. 4, comprises a horizontal direction moving mechanism part (not shown) which can be moved in an X2 axis direction and a Y2 axis direction orthogonal in a horizontal direction, a specimen·latex pipette part 211 which can be moved in a direction (Z2 axis direction) vertical to the horizontal direction moving mechanism part, and a plate catcher part 212. In addition, the specimen·latex pipette part 211 has the function of dispensing and discharging a sample. In addition, the specimen·latex pipette part 211 also has the function of dispensing and discharging a latex reagent, a buffer and a specimen diluting solution in a reagent bin 203. In addition, the plate catcher part 212 is provided for conveying the unused reaction plate 201 from the reaction plate tray 280 to the reaction part 240 and, at the same time, conveying a used reaction plate 201 to a reaction plate discarding case (not shown). In the reaction plate 201, 25 cuvettes 201a which can accommodate a sample and various reagents are provided.

The reagent setting part 220 is provided for mounting a reagent bin 203 accommodating a buffer, a latex reagent and a specimen diluting solution. Thereupon, reagents (buffer, latex reagent, specimen diluting solution) in the reagent bin 203 are maintained at a predetermined temperature (not higher than, 15° C.). In addition, in the reagent setting part 220, a buffer container setting part 221, a latex reagent container setting part 220 and a specimen diluting solution container setting part 223 are provided in an order from an inner side of the apparatus.

The reaction part 240 is provided for reacting a sample accommodated in a cuvette 201a in two reaction plates 201, and various reagents (buffer, latex reagent containing latex particle obtained by sensitizing anti-CK19 antibody, specimen diluting solution). Specifically, by stirring and mixing a sample dispensed from the dispensing part 210, and various reagents (buffer, latex reagent, specimen diluting solution) and, at the same time, maintaining the stirred and mixed sample and various reagents at a predetermined temperature, a reaction of aggregating the latex reagent is promoted. That is, at this reaction part 240, as shown in FIG. 5, an aggregation reaction of aggregating latex particles in the latex reagent with an antibody bound thereto via an antigen (CK19 protein) in the sample is performed.

When a CK19 protein is present in a sample, as shown in a right figure of FIG. 5, a plurality of latex particles obtained by sensitizing the CK19 protein with an anti-CK19 antibody by an antigen-antibody reaction are bound, and particle aggregation occurs. Since as an amount of the CK19 protein is greater, particles aggregate more, the CK19 protein is quantitated by measuring an aggregation degree of a sample by the CIA.

The measurement dilution dispensing part 250, as shown in FIG. 4, is arranged rear the dispensing part 210, and has the function of sucking and discharging a prepared sample in the cuvette 201a in the reaction plate 201 of the reaction part 240. This measurement dilution dispensing part 250 comprises a horizontal direction moving mechanism part (not shown) which can be moved in an X2 axis direction and a Y2 axis direction orthogonal in a horizontal direction, and a measurement dilution pipette part 251 which can be moved in a direction (Z2 axis direction) vertical to the horizontal direction moving mechanism part. In addition, the measurement dilution dispensing part 250 discharges the sucked prepared sample in the cuvette 201a of the reaction plate 201 together with a measurement diluting solution accommodated in a tank (not shown) disposed at a lower part of an immunoagglutination measurement device 200, to a sample receiving part 260.

The sample receiving part 260 is provided for receiving the prepared sample and the measurement diluting solution in the cuvette 201a of the reaction plate 201 of the reaction part 240. In addition, a particle suspension (prepared sample and measurement diluting solution) received in the sample receiving part 260 is guided to a sheath flow cell 274 (see FIG. 6) at an optical detection part 270 described later.

The optical detection part 270, as shown in FIG. 6, is constructed of a laser diode 271 as a light source, a condenser lens 272 and a collector lens 273, a sheath flow cell 274, and a photodiode 275 as a light receiving element. The sheath flow cell 274 has the function of converting a stream of the particle suspension (prepared sample and measurement diluting solution) into a flat stream by holding the stream by sheath solution streams flowing on both sides of the particle suspension. In addition, light irradiated to the particle suspension flowing in the sheath flow cell 274 from the laser diode 271 is scattered by an aggregated mass (see FIG. 5) of the latex particles in the particle suspension, to be received by the photodiode 275. Information of scattered light received by the photodiode 275 is sent to the personal computer 102.

The reaction plate tray 280 can accommodate a maximum of four unused reaction plates 201 (see FIG. 4). In addition, the reaction plate 201 accommodated in the reaction plate tray 280 is conveyed to the reaction part 240 by the plate catcher part 212 (see FIG. 4) of the dispensing part 210. In addition, the reaction plate discarding case can store a used reaction plate 201, and is conveyed from the reaction part 240 by a plate catcher of the dispensing part 210.

The washing part 300a is provided for washing the specimen·latex pipette part 211 of the dispensing part 210. In addition, the washing part 300b is provided for washing the measurement dilution pipette part 251 of the measurement dilution dispensing part 250.

The CPU 102d of the personal computer 102 acquires scattered light information sent from the photodiode 275 and, based on this, calculates an amount of the CK19 protein in a sample. The CPU 102d compares the calculated CK19 protein quantitated value with a predetermined corresponding threshold.

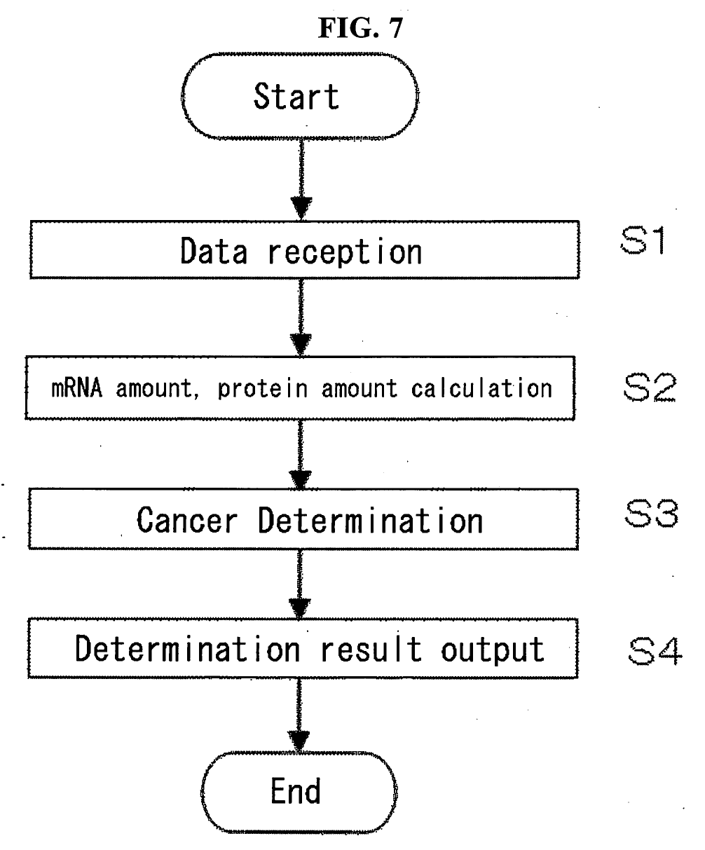

A process flow of the CPU 102d will be explained based on FIG. 7. As described above, the CPU 102d receives turbidity data from the nucleic acid quantitation part 100, and scattered light information from the protein quantitation part 101 (step S1). Based on the turbidity data, an expression amount of an mRNA is calculated and, based on scattered light information, an expression amount of a protein is calculated (step S2). Then, the CPU 102d determines the presence or absence of a cancer cell in a sample by comparing a CK19 mRNA quantitated value and a predetermined threshold, and comparing a CK19 protein quantitated value with a predetermined threshold (step S3). The CPU 102d outputs and displays the determination result on the display part 102c of the personal computer 102.

The nucleic acid quantitation part 100 and the protein quantitation part 101 maybe separate devices (i.e. nucleic acid quantitation device and protein quantitation device).

In addition, since the nucleic acid quantitation part 100 and the protein quantitation part 101 use the same sample, a conveying device for conveying this sample to each quantitation part may be provided.

### EXAMPLE

A result of a method for detecting a cancer which actually uses the kit for detecting a cancer cell of the present invention will be specifically explained below by way of Example.

### Example 1

### 1. Preparation of Sample for Measurement

Using 31 lymph nodes excised from a breast cancer patient, samples for measurement were prepared. Among them, by microscopy of a hematoxylin-eosin stained tissue section, metastasis of a cancer cell was recognized in 22 lymph nodes, and metastasis of a cancer cell was not recognized in 9 lymph nodes.

Using 22 lymph nodes for which metastasis of a cancer cell was histologically recognized (positive lymph node), and 9 lymph nodes for which metastasis of a cancer cell was not histologically recognized (negative lymph node), a sample for measurement was prepared as follows.

First, 200 μL of a buffer (containing 200 mM glycine-HCl 5% Brij35(polyoxyethylene(35)lauryl ether, manufactured by SIGMA CORPORATION) and 20% DMSO (Wako Pure Chemical Industries, Ltd.)) of a pH of 3.4 was added to each lymph node (about 50 to 600 mg/node), and this was homogenized with a blender. The resulting homogenate was centrifuged at 10,000×g and room temperature for 1 minute, and 50 to 200 μL of a supernatant was collected, which was used as a sample for detection.

### 2. Quantitation of CK19 mRNA

Samples for detection from positive lymph nodes and negative lymph nodes obtained as described above were set on a nucleic acid amplification apparatus (GD-100, manufactured by Sysmex Corporation), and a CK19cDNA was amplified by an RT-LAMP reaction (as a reagent for measurement, a reagent for nucleic acid amplification, a cytokeratin reagent (manufactured by Sysmex Corporation) was set in GD-100). By measuring a turbidity of the reaction solution in real time, an mRNA of CK19 (copy number) was quantitated.

### 3. Quantitation of CK19 protein

To 20 μL of the sample for detection used in quantitation of the CK19 mRNA was added 10 μL of a 3×SDS treatment buffer (containing 150 mM Tris HCl (pH 6.8), 300 mM dithiothreitol, 6% sodium dodecylsulfate (SDS), 0.3% bromophenol blue, and 30% glycerol), and the mixture was warmed at 95° C. for 5 minutes to prepare a sample for electrophoresis. Using 15 μL of this sample for electrophoresis, SDS-PAGE was performed. A gel after electrophoresis was transferred to a PVDF membrane, this was immersed in a blocking buffer (containing 20 mM Tris (pH 7.6), 137 mM NaCl, 0.1% Tween20, and 5% skim milk), and shaken at room temperature for 1 hour, thereby, the membrane was subjected to blocking treatment. After the blocking treatment, the membrane was washed with TBS-T (20 mM Tris (pH 7.6), 137 mM NaCl, and 0.1% Tween20) for 2 minutes. Then, the membrane was immersed in a primary antibody solution (solution obtained by 1/500 diluting a primary antibody: Cytokeratin 19 (A53-B/A2):sc-6278 (manufactured by Santa Cruz Biotechnology, Inc., Lot:#L2403) with TBS-T), and allowed to stand at 4° C. overnight to perform an antibody reaction. After the reaction, this membrane was washed with TBS-T for 5 minutes four times, and the membrane was immersed in a solution of a secondary antibody which can bound to the primary antibody and is bound to horseradish peroxidase as a labeling enzyme (solution obtained by 1/2000 diluting a secondary antibody: ECL anti-mouse IgG HRP linked F(ab′2) fragment (manufactured by GE Health Care Biosciences) with TBS-T), and allowed to stand at room temperature for 30 minutes, to perform an antibody reaction. After the reaction, the membrane was washed with TBS-T for 5 minutes four times, and an enzyme reaction was performed with ECL-Advance Western Blotting Detection Kit (manufactured by GE Health Care Biosciences). A membrane image after the enzyme reaction was taken with LumiAnalyst (manufactured by Roche Diagnostics), and a fluorescent intensity of the membrane image was calculated with Quantityone (manufactured by Bio-Rad Laboratories). The fluorescent intensity measured as described above was fitted with a calibration curve to calculate an amount of the CK19 protein. The calibration curve was produced by accommodating a sample containing the CK19 protein having a known concentration in a well other than a well accommodating a sample for measurement with the same gel, and performing the aforementioned fluorescent intensity measurement.

### 4. Result

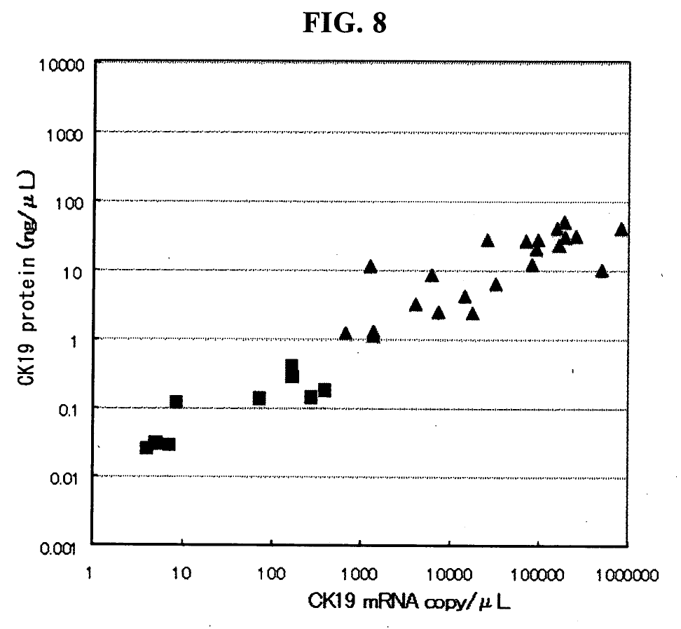

The measured mRNA quantitated value (copy/μL) and protein quantitated value (ng/μL) were developed on a logarithmic axis to produce a graph. This graph is shown in FIG. 8. In the graph, ▴ is a positive sample, and ▪ is a negative sample. As shown in FIG. 8, the quantitation result of an mRNA and the quantitation result of a protein showed a good correlation. Therefore, by using these quantitation results, whether a biological sample subjected to measurement is a negative sample or a positive sample can be determined.

In addition, a first threshold corresponding to an mRNA quantitated value can be 5000 copy/μL, and a second threshold corresponding to a protein quantitated value can be 0.5 ng/μL. By comparing the mRNA quantitated value and the protein quantitated value with these thresholds, the presence or absence of cancer metastasis may be determined.

### Example 2.

### 1. Preparation of Sample for Measurement

Using 63 lymph nodes excised from a colon cancer patient, samples for measurement were prepared. Among them, by microscopy of a hematoxylin-eosin stained tissue section, metastasis of a cancer cell was recognized in 35 lymph nodes, and metastasis of a cancer cell was not recognized in 28 lymph nodes.

Using 35 lymph nodes for which metastasis of a cancer cell was histologically recognized (positive lymph node), and 28 lymph nodes for which metastasis of a cancer cell was not histologically recognized (negative lymph node), a sample for measurement was prepared as in Example 1.

### 2. Quantitation of CK19 mRNA and CK19 Protein

Using the above samples for detection, the CK19 mRNA and CK19 protein were quantitated as in Example 1.

### 3. Result

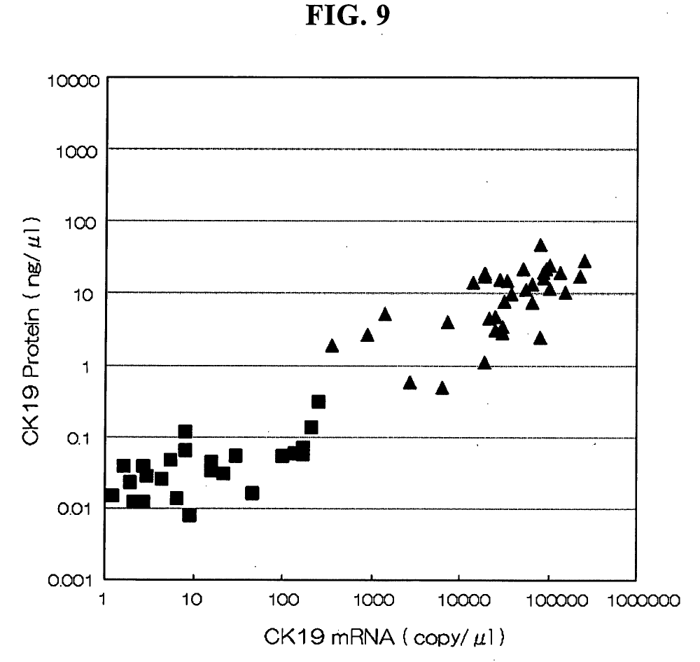

The measured mRNA quantitated value (copy/μL) and protein quantitated value (ng/μL) were developed on a logarithmic axis to produce a graph. This graph is shown in FIG. 9. In the graph, ▴ is a positive sample, and ▪ is a negative sample. As shown in FIG. 9, the quantitation result of an mRNA and the quantitation result of a protein also showed a good correlation in cases using a colon cancer.

### Example 3.

### 1. Preparation of Sample for Measurement

Using 30 lymph nodes excised from a stomach cancer patient, samples for measurement were prepared. Among them, by microscopy of a hematoxylin-eosin stained tissue section, metastasis of a cancer cell was recognized in 24 lymph nodes, and metastasis of a cancer cell was not recognized in 6 lymph nodes.

Using 24 lymph nodes for which metastasis of a cancer cell was histologically recognized (positive lymph node), and 6 lymph nodes for which metastasis of a cancer cell was not histologically recognized (negative lymph node), a sample for measurement was prepared as in Example 1.

### 2. Quantitation of CK19 mRNA and CK19 Protein

Using the above samples for detection, the CK19 mRNA and CK19 protein were quantitated as in Example 1.

### 3. Result

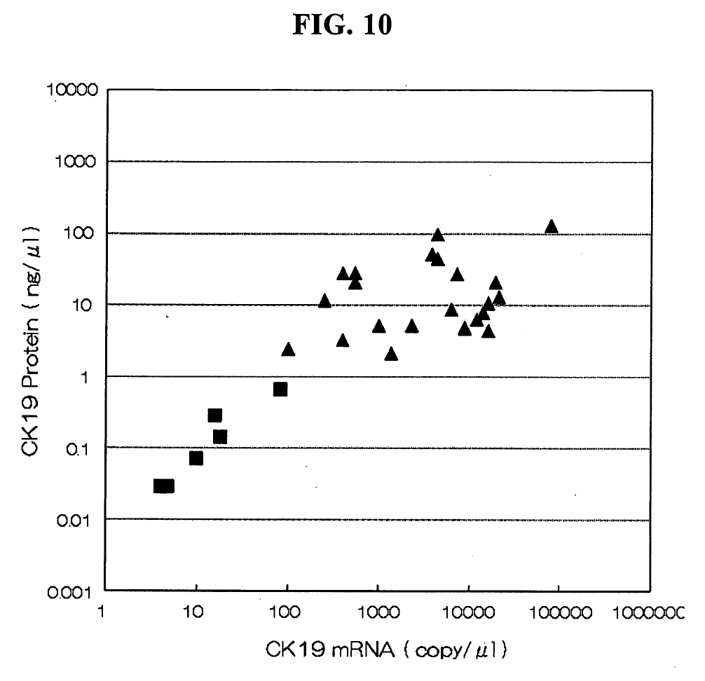

The measured mRNA quantitated value (copy/μL) and protein quantitated value (ng/μL) were developed on a logarithmic axis to produce a graph. This graph is shown in FIG. 10. In the graph, ▴ is a positive sample, and ▪ is a negative sample. As shown in FIG. 10, the quantitation result of an mRNA and the quantitation result of a protein also showed a good correlation in cases using a stomach cancer.

Since the present Example is a detection method based on the mRNA quantitation result and the protein quantitation result, a detection result which has better precision and higher reliability than those of a detection result by a single method is obtained.

The foregoing detailed description and example have been provided by way of explanation and illustration, and are not intended to limit the scope of the appended claims. Many variations in the presently preferred embodiments will be obvious to one of ordinary skill in the art, and remain within the scope of the appended claims and their equivalents.

What is claimed is:

1. A method for detecting a cancer cell, comprising steps of:
quantitating an mRNA transcribed from a tumor marker gene contained in a biological sample,
quantitating a polypeptide translated from the tumor marker gene in the biological sample, and
detecting a cancer cell in the biological sample based on the quantitation result of the mRNA and the quantitation result of the polypeptide.

2. The method according to claim 1, wherein the biological sample is a lymph node tissue, and the quantitation steps of the mRNA and the polypeptide are performed so as to use a sample for detection prepared from the biological sample.

3. The method according to claim 2, wherein the quantitation steps of the mRNA and the polypeptide are performed so as to use the same sample for detection.

4. The method according to claim 2, wherein the sample for detection is a solution obtained by treating the lymph node tissue in a buffer to transfer an RNA and a polypeptide in the tissue into a solution.

5. The method according to claim 4, wherein the buffer has a pH of 2.5 to 5.0.

6. The method according to claim 4, wherein the buffer comprises dimethyl sulfoxide.

7. The method according to claim A, wherein the buffer comprises a surfactant.

8. The method according to claim 7, wherein the surfactant is a nonionic surfactant.

9. The method according to claim 7, wherein the surfactant is a polyoxyethylene nonionic surfactant.

10. The method according to claim 1, wherein the tumor marker is cytokeratin.

11. The method according to claim 1, wherein the cancer is carcinoma.

12. The method according to claim 1, wherein the cancer is a breast cancer, a stomach cancer or a colon cancer.

13. A method for preparing a sample for detection for detecting a nucleic acid and a polypeptide, comprising a step of:
treating a biological sample using a buffer containing dimethyl sulfoxide and a surfactant and having a pH of 2.5 to 5.0,
thereby, transferring a nucleic acid and a polypeptide contained in the biological sample into a solution to prepare a sample for detection.

14. The method according to claim 13, wherein the biological sample is a lymph node tissue.

15. The method according to claim 13, wherein the surfactant is a nonionic surfactant.

16. The method according to claim 13, wherein the surfactant is a polyoxyethylene nonionic surfactant.

17. An apparatus for detecting a cancer cell, comprising:
a first quantitation part for quantitating an mRNA transcribed from a tumor marker gene contained in a biological sample,
a second quantitation part for quantitating a polypeptide translated from the tumor marker gene contained in the biological sample, and
a determination part for detecting a cancer cell in the biological sample based on the quantitation result of the mRNA and the quantitation result of the polypeptide.

18. The detection apparatus according to claim 17,
wherein the tumor marker gene is cytokeratin.

19. The detection apparatus according to claim 17, wherein the cancer is carcinoma.

20. The detection apparatus according to claim 17, wherein the cancer is a breast cancer.
